(19) United States
(12) Patent Application Publication
(10) Pub. No.: US 2016/0060552 A1
(43) Pub. Date: Mar. 3, 2016

(54) POLYALKYL SUCCINIC ANHYDRIDE DERIVATIVES AS ADDITIVES FOR FOULING MITIGATION IN PETROLEUM REFINERY PROCESSES

(71) Applicants: Man Kit Ng, Basking Ridge, NJ (US); Glen Barry Brons, Leland, NC (US); Steven Webster Levine, Flemington, NJ (US); Kevin Mallory, Clinton, NJ (US); Hong Cheng, Bridgewater, NJ (US); Patrick Brant, Seabrook, TX (US); Donna J. Crowther, Seabrook, TX (US)

(72) Inventors: Man Kit Ng, Basking Ridge, NJ (US); Glen Barry Brons, Leland, NC (US); Steven Webster Levine, Flemington, NJ (US); Kevin Mallory, Clinton, NJ (US); Hong Cheng, Bridgewater, NJ (US); Patrick Brant, Seabrook, TX (US); Donna J. Crowther, Seabrook, TX (US)

(73) Assignee: ExxonMobil Research and Engineering Company, Annandale, NJ (US)

(21) Appl. No.: 14/939,361

(22) Filed: Nov. 12, 2015

Related U.S. Application Data
(62) Division of application No. 12/642,453, filed on Dec. 18, 2009.
Application No. 12/642,453 is a continuation-in-part of application No. 12/533,465, filed on Jul. 31, 2009, now abandoned.
(60) Provisional application No. 61/136,172, filed on Aug. 15, 2008.

(51) Int. Cl. C10G 75/04; C07D 307/60; C09K 15/30

(52) U.S. Cl. CPC C10G 75/04; C09K 15/30; C07D 307/60

(57) ABSTRACT

The present invention provides a method for reducing fouling, including particulate-induced fouling, in a hydrocarbon refining process including the steps of providing a crude hydrocarbon for a refining process; adding at least one polyalkyl succinic anhydride derivative additive disclosed herein. The additive can be complexed with a boronating agent, such as boric acid, to yield a boron-containing polyalkyl succinic anhydride derivative.

### CROSS REFERENCE TO RELATED APPLICATIONS

The present application is a continuation-in-part application of U.S. Utility application Ser. No. 12/533,465 filed on Jul. 31, 2009, which claims priority to U.S. Provisional Application Ser. No. 61/136,172, filed on Aug. 15, 2008, the disclosures of both of which are incorporated by reference herein in their entirety.

This invention is also related to U.S. Utility application Ser. No. 12/488,066, filed Jun. 19, 2009, U.S. Utility application Ser. No. 12/488,093, filed Jun. 19, 2009, U.S. Utility application Ser. No. 12/487,739, filed Jun. 19, 2009, and U.S. Utility application Ser. No. 12/143,663 filed Jun. 20, 2008, the disclosures of each of which are incorporated by reference herein in their entirety.

### FIELD OF THE INVENTION

The present invention relates to additives to reduce fouling of crude hydrocarbon refinery components, and methods and systems using the same.

### BACKGROUND OF THE INVENTION

Petroleum refineries incur additional energy costs, perhaps billions per year, due to fouling and the resulting attendant inefficiencies caused by the fouling. More particularly, thermal processing of crude oils, blends and fractions in heat transfer equipment, such as heat exchangers, is hampered by the deposition of insoluble asphaltenes and other contaminants (i.e., particulates, salts, etc.) that may be found in crude oils. Further, the asphaltenes and other organics may thermally degrade to coke when exposed to high heater tube surface temperatures.

Fouling in heat exchangers receiving petroleum-type process streams can result from a number of mechanisms including chemical reactions, corrosion, deposit of existing insoluble impurities in the stream, and deposit of materials rendered insoluble by the temperature difference (ΔT) between the process stream and the heat exchanger wall. For example, naturally-occurring asphaltenes can precipitate from the crude oil process stream, thermally degrade to form a coke and adhere to the hot surfaces. Further, the high ΔT found in heat transfer operations result in high surface or skin temperatures when the process stream is introduced to the heater tube surfaces, which contributes to the precipitation of insoluble particulates. Another common cause of fouling is attributable to the presence of salts, particulates and impurities (e.g. inorganic contaminants) found in the crude oil stream. For example, iron oxide/sulfide, calcium carbonate, silica, sodium chloride and calcium chloride have all been found to attach directly to the surface of a fouled heater rod and throughout the coke deposit. These solids promote and/or enable additional fouling of crude oils.

The buildup of insoluble deposits in heat transfer equipment creates an unwanted insulating effect and reduces the heat transfer efficiency. Fouling also reduces the cross-sectional area of process equipment, which decreases flow rates and desired pressure differentials to provide less than optimal operation. To overcome these disadvantages, heat transfer equipment are ordinarily taken offline and cleaned mechanically or chemically cleaned, resulting in lost production time.

Accordingly, there is a need to reduce precipitation/adherence of particulates and asphaltenes from the heated surface to prevent fouling, and before the asphaltenes are thermally degraded or coked. This will improve the performance of the heat transfer equipment, decrease or eliminate scheduled outages for fouling mitigation efforts, and reduce energy costs associated with the processing activity.

### SUMMARY OF THE INVENTION

One aspect of the present invention provides a method for reducing fouling in a hydrocarbon refining process. The method includes providing a crude hydrocarbon for a refining process; and adding to the crude hydrocarbon one or more additives selected from:

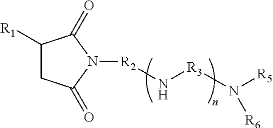

wherein R₁ is a branched or straight-chained C₁₀-C₈₀₀ alkyl or alkenyl group;
n is an integer from 1 to 10;
R₂ and R₃ are independently a C₁-C₁₀ branched or straight chained alkylene group;

### R₅ and R₆ are H, or

R₅ and R₆ together along with the N atom bound thereto form the group:

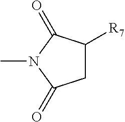

wherein R₇ is a branched or straight-chained C₁₀-C₈₀₀ alkyl or alkenyl group;
wherein the N atom bound to the R₂ and R₃ groups above is optionally substituted in one or more places with the group:

—R₈—R₉

wherein R₈ is a C₁-C₁₀ branched or straight chained alkylene group; and R₉ is NH₂ or

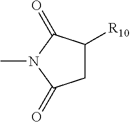

wherein R₁₀ is a branched or straight-chained C₁₀-C₈₀₀ alkyl or alkenyl group; and wherein the R₂—NH—R₃ group is optionally interrupted in one or more places by a heterocyclic or homocyclic cycloalkyl group. In one particular embodiment, the fouling is particulate-induced fouling.

In certain embodiments, one or more of the R₁, R₇ and R₁₀ groups is independently selected from polypropylene (PP) and poly(ethylene-co-propylene) (EP). The polypropylene and poly(ethylene-co-propylene) groups themselves can contain from about 10 to about 800 carbon atoms and can have a number average molecular weight (Mn) up to and exceeding 10,000 g/mol (assuming one olefin unsaturation per chain, as measured by ¹H NMR), i.e., one or more of the R₁, R₇ and R₁₀ groups can individually have a Mn up to and exceeding 10,000 g/mol. In one embodiment, for example, the PP or EP groups, individually, have a Mn from about 300 to about 30,000 g/mol, or from about 500 to about 5000 g/mol.

Another aspect of the present invention is directed to a system for refining hydrocarbons. The system includes at least one crude hydrocarbon refinery component and crude hydrocarbon in fluid communication with the at least one crude hydrocarbon refinery component, wherein the crude hydrocarbon includes at least one of any of the additives described herein. In one particular embodiment, the system is particularly adept at reducing and/or preventing particulate-induced fouling, such as particulate induced fouling in a petroleum refining operation.

Another aspect of the present invention provides a composition for reducing fouling (e.g. particulate-induced fouling) that includes at least one of the above-described additives, and a boronating agent complexed or in association with any one of the above-mentioned additives.

### BRIEF DESCRIPTION OF THE DRAWINGS

The invention will now be described in conjunction with the accompanying drawings in which:

FIG. 1 is a representation of an oil refinery crude pre-heat train, annotated to show non-limiting injection points for the additives of the present invention.

FIG. 2 is a schematic of the Alcor Hot Liquid Process Simulator (HLPS) employed in Example 2 of this invention, with an illustrative data plot.

FIG. 3 is a graph demonstrating the effects of fouling of a crude oil stream and a crude oil stream treated with 250 wppm of a polyisobutyl succinic acid-polyamine ester, as measured by the Alcor HLPS apparatus depicted in FIG. 2.

FIG. 4 is a graph demonstrating the reduction of fouling achieved by two non-borate containing additives and one borate containing additive of the present invention, as compared to a control stream containing no additive, as measured by the Alcor HLPS apparatus depicted in FIG. 2.

FIG. 5 is a graph demonstrating the effects of fouling, as measured by the Alcor HLPS apparatus depicted in FIG. 2, of control crude oil blend samples and a crude oil blend sample containing a PP-SA-PAM additive having a total nitrogen content of 5.83 wt % and a propylene chain having a Mn of about 571 g/mol. Molecular weights are based on ¹H NMR analysis of the allylic vinyl-terminated polypropylene starting materials prior to the maleation reaction (assuming one olefin unsaturation per chain), as that synthesis step is described in Example 1 below.

FIG. 6 is a graph demonstrating the effects of fouling, as measured by the Alcor HLPS apparatus depicted in FIG. 2, of control crude oil blend samples and a crude oil blend sample containing a PP-SA-PAM additive having a total nitrogen content of 5.70 wt % and a propylene chain having a Mn of about 945 g/mol. Molecular weights are based on 1H NMR analysis of the allylic vinyl-terminated polypropylene starting materials prior to the maleation reaction (assuming one olefin unsaturation per chain), as that synthesis step is described in Example 1 below.

FIG. 7 is a graph demonstrating the effects of fouling, as measured by the Alcor HLPS apparatus depicted in FIG. 2, of control crude oil blend samples and a crude oil blend sample containing a PP-SA-PAM additive having a total nitrogen content of 2.33 wt % and a propylene chain having a Mn of about 2,332 g/mol. Molecular weights are based on ¹H NMR analysis of the allylic vinyl-terminated polypropylene starting materials prior to the maleation reaction (assuming one olefin unsaturation per chain), as that synthesis step is described in Example 1 below.

FIG. 8 is a graph demonstrating the effects of fouling as measured by the Alcor HLPS apparatus depicted in FIG. 2, of control crude oil blend samples and a crude oil blend sample containing a PP-SA-PAM additive having a total nitrogen content of 3.38 wt % and a propylene chain having a Mn of about 2,332 g/mol. Molecular weights are based on ¹H NMR analysis of the allylic vinyl-terminated polypropylene starting materials prior to the maleation reaction (assuming one olefin unsaturation per chain), as that synthesis step is described in Example 1 below.

FIG. 9 is a graph demonstrating the effects of fouling, as measured by the Alcor HLPS apparatus depicted in FIG. 2, of control crude oil blend samples and a crude oil blend sample containing a EP-SA-PAM additive having a total nitrogen content of 8.42 wt % and a poly(ethylene-co-propylene) chain having a Mn of about 828 g/mol. Molecular weights are based on ¹H NMR analysis of the allylic vinyl-terminated poly(ethylene-co-propylene) starting materials prior to the maleation reaction (assuming one olefin unsaturation per chain), as that synthesis step is described in Example 1 below.

### DETAILED DESCRIPTION OF THE INVENTION

### Definitions

The following definitions are provided for purpose of illustration and not limitation.

As used herein, the term “fouling” generally refers to the accumulation of unwanted materials on the surfaces of processing equipment or the like.

As used herein, the term “particulate-induced fouling” generally refers to fouling caused primarily by the presence of variable amounts of organic or inorganic particulates. Organic particulates (such as precipitated asphaltenes and coke particles) include, but are not limited to, insoluble matter precipitated out of solution upon changes in process conditions (e.g. temperature, pressure, or concentration changes) or a change in the composition of the feed stream (e.g. changes due to the occurrence of a chemical reaction). Inorganic particulates include, but are not limited to, silica, iron oxide, iron sulfide, alkaline earth metal oxide, sodium chloride, calcium chloride and other inorganic salts. One major source of these particulates results from incomplete solids removal during desalting and/or other particulate removing processes. Solids promote the fouling of crude oils and blends due to physical effects by modifying the surface area of heat transfer equipment, allowing for longer holdup times at wall temperatures and causing coke formation from asphaltenes and/or crude oil(s).

As used herein, the term “alkyl” refers to a monovalent hydrocarbon group containing no double or triple bonds and arranged in a branched or straight chain.

As used herein, the term “alkylene” refers to a divalent hydrocarbon group containing no double or triple bonds and arranged in a branched or straight chain.

As used herein, the term “alkenyl” refers to a monovalent hydrocarbon group containing one or more double bonds and arranged in a branched or straight chain.

As used herein, the abbreviation “PIB” refers to polyisobutylene and includes both normal or “conventional” polyisobutylene and highly reactive polyisobutylene (HRPIB).

As used herein and as would be understood by persons of ordinary skill in the art, reference to a group being a particular polymer (e.g., polypropylene, poly(ethylene-co-propylene) or PIB) encompasses polymers that contain primarily the respective monomer along with negligible amounts of other substitutions and/or interruptions along polymer chain. In other words, reference to a group being a polypropylene group does not require that the group consist of 100% propylene monomers without any linking groups, substitutions, impurities or other substituents (e.g. alkylene or alkenylene substituents). Such impurities or other substituents may be present in relatively minor amounts so long as they do not affect the industrial performance of the additive, as compared to the same additive containing the respective polymer substituent with 100% purity.

As used herein, a “boronating agent” include compounds encompassed by the formula:

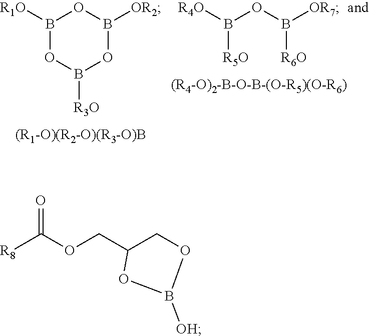

wherein R₁, R₂, R₃, R₄, R₅, R₆, R₇ and R₈ are independently C₃ to C₂₀ hydrocarbyl groups. Examples of these materials include Mobilad™ C-700 and Mobilad™ C-701, available from ExxonMobil Chemical Co., Houston, Tex.).

As used herein, a “boronating agent” also includes compounds disclosed in International Published Application No. PCT/US96/13618 (published as WO/1997/011220), applied for by Mobil Oil Corporation and hereby incorporated by reference in its entirety. Accordingly, boric acid can be used as a boronating agent: organic borates, particularly ortho-borates, meta-borates, trialkyl borates can also be used in additive-containing compositions of the present invention. Suitable metaborates include, but are not limited to, trimethyl metaborate (trimethoxyboroxine), triethyl metaborate, tributyl metaborate. Suitable trialkyl borates include, without limitation, trimethyl borate, triethylborate, triisopropyl borate (triisopropoxyborane), tributyl borate (tributoxyborane) and tri-t-butyl borate.

As used herein, a “hydrocarbyl” group refers to any univalent radical that is derived from a hydrocarbon, including univalent alkyl, aryl and cycloalkyl groups.

As used herein, the term “crude hydrocarbon refinery component” generally refers to an apparatus or instrumentality of a process to refine crude hydrocarbons, such as an oil refinery process, which is, or can be, susceptible to fouling. Crude hydrocarbon refinery components include, but are not limited to, heat transfer components such as a heat exchanger, a furnace, a crude preheater, a coker preheater, or any other heaters, a FCC slurry bottom, a debutanizer exchanger/tower, other feed/effluent exchangers and furnace air preheaters in refinery facilities, flare compressor components in refinery facilities and steam cracker/reformer tubes in petrochemical facilities. Crude hydrocarbon refinery components can also include other instrumentalities in which heat transfer can take place, such as a fractionation or distillation column, a scrubber, a reactor, a liquid-jacketed tank, a pipestill, a coker and a visbreaker. It is understood that “crude hydrocarbon refinery components,” as used herein, encompasses tubes, piping, baffles and other process transport mechanisms that are internal to, at least partially constitute, and/or are in direct fluid communication with, any one of the above-mentioned crude hydrocarbon refinery components.

As used herein, a reduction (or “reducing”) particulate-induced fouling is generally achieved when the ability of particulates to adhere to heated equipment surfaces is reduced, thereby mitigating their impact on the promotion of the fouling of crude oil(s), blends, and other refinery process streams.

For the purposes of this invention and the claims thereto when a polymer is referred to as comprising an olefin, the olefin present in the polymer is the polymerized form of the olefin.

As used herein, a copolymer is an polymer comprising at least two different monomer units (such as propylene and ethylene). A homo-polymer is an polymer comprising units of the same monomer (such as propylene). A propylene polymer is a polymer having at least 50 mole % of propylene.

The term “vinyl termination”, also referred to as “allyl chain end(s)” or “vinyl content” is defined to be a polymer having at least one terminus represented by formula I:

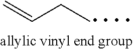

where the “••••” represents the polymer chain.

In a preferred embodiment the allyl chain end is represented by the formula II:

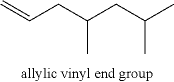

The amount of allyl chain ends (also called % vinyl termination) is determined using ¹H NMR at 120° C. using deuterated tetrachloroethane as the solvent on a 500 MHz machine and in selected cases confirmed by ¹³C NMR. Resconi has reported proton and carbon assignments (neat perdeuterated tetrachloroethane used for proton spectra while a 50:50 mixture of normal and perdeuterated tetrachloroethane was used for carbon spectra; all spectra were recorded at 100° C. on a Bruker AM 300 spectrometer operating at 300 MHz for proton and 75.43 MHz for carbon) for vinyl terminated propylene polymers in J American Chemical Soc 114 1992, 1025-1032, hereby incorporated by reference in its entirety, that are useful herein.

“Isobutyl chain end” is defined to be a polymer having at least one terminus represented by the formula:

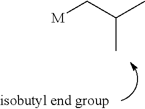

where M represents the polymer chain. In a preferred embodiment, the isobutyl chain end is represented by one of the following formulae:

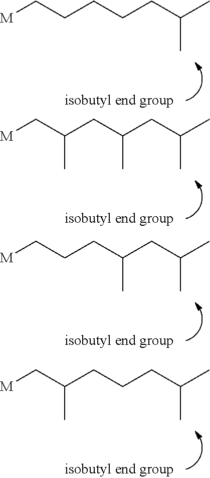

where M represents the polymer chain.

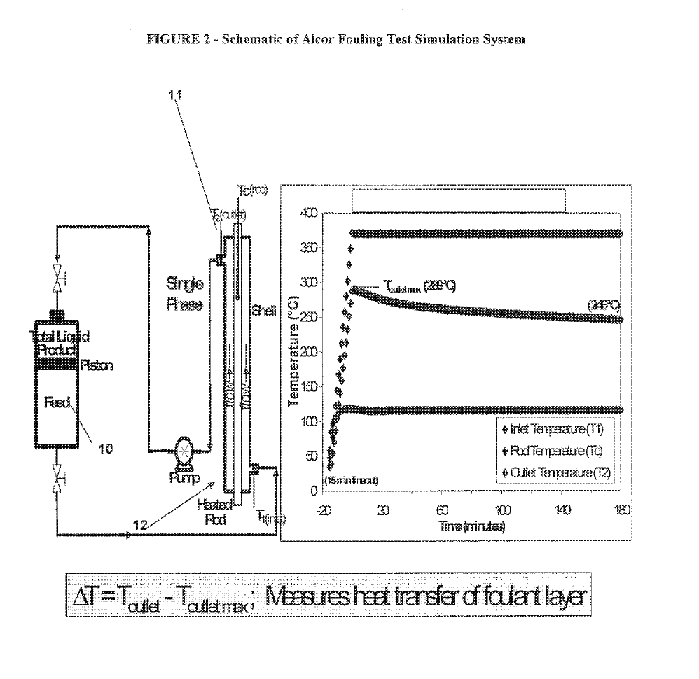

The percentage of isobutyl end groups is determined using ¹³C NMR (as described in the example section of Ser. No. 12/488,066, filed Jun. 19, 2009) and the chemical shift assignments in Resconi et al, J Am. Chem. Soc. 1992, 114, 1025-1032 for 100% propylene polymers and set forth in FIG. 2 for E-P polymers.

The “isobutyl chain end to allylic vinyl group ratio” is defined to be the ratio of the percentage of isobutyl chain ends to the percentage of allylic vinyl groups.

A reaction zone is any vessel where a reaction occurs, such as glass vial, a polymerization reactor, reactive extruder, tubular reactor and the like.

As used herein, the term “polymer” refers to a chain of monomers having a Mn of 100 g/mol and above.

Reference will now be made to various aspects of the present invention in view of the definitions above.

The techniques provided herein are based, at least in part, on interactions between the antifouling additives according to the invention and the materials in crude oils that are prone to cause fouling, e.g., particulate impurities/contaminants and asphaltenes. The interaction can be of physical or chemical means such as absorption, association, or chemical bonding. The fouling materials can be rendered more soluble in the crude oils as a result of interaction with the antifouling additives, therefore the fouling on the exchanger metal surfaces can be reduced or eliminated.

In accordance with one aspect of the present invention, a method is provided for reducing fouling in which one or more additives are selected from:

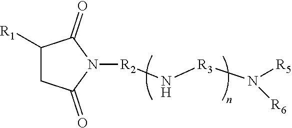

wherein R₁ is a branched or straight-chained C₁₀-C₈₀₀ alkyl or alkenyl group;
n is an integer from 1 to 10;
R₂ and R₃ are independently a C₁-C₁₀ branched or straight chained alkylene group;
R₅ and R₆ are H, or R₅ and R₆ together along with the N atom bound thereto form the group:

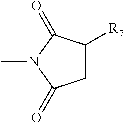

wherein R₇ is a branched or straight-chained C₁₀-C₈₀₀ alkyl or alkenyl group; wherein the N atom bound to the R₂ and R₃ groups above is optionally substituted in one or more places with the group:

—R₈—R₉

wherein R₈ is a C₁-C₁₀ branched or straight chained alkylene group; and R₉ is NH₂ or

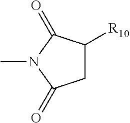

wherein R₁₀ is a branched or straight-chained C₁₀-C₈₀₀ alkyl or alkenyl group; and wherein the R₂—NH—R₃ group is optionally interrupted in one or more places by a heterocyclic or homocyclic cycloalkyl group. The additive can be added to a crude hydrocarbon process stream in a variety of locations and manners as described in order to reduce various types of fouling. In one particular embodiment, the fouling is particulate-induced fouling.

In certain embodiments, one or more of the R₁, R₇ and R₁₀ groups is independently selected from polypropylene (PP) and poly(ethylene-co-propylene) (EP). The polypropylene and poly(ethylene-co-propylene) groups themselves can contain from about 10 to about 800 carbon atoms and can have a Mn up to and exceeding 10,000 g/mol, i.e., one or more of the R₁, R₇ and R₁₀ groups can individually have a molecular weight up to and exceeding 10,000 g/mol. As used above, number averaged molecular weights (Mn) are based on the assumption of one olefin unsaturation per chain, as measured by ¹H NMR.

In one embodiment, the PP or EP groups, individually, have a Mn from about 300 to about 30,000 g/mol, or from about 500 to about 5000 g/mol. In one embodiment, the PP or EP groups (i.e., one or more of the R₁, R₇ and R₁₀ groups) have a Mn, individually, ranging from about 500 to about 2500 g/mol, or a molecular weight of from about 500 to about 650 g/mol or a molecular weight of from about 800 to about 1000 g/mol, or a molecular weight of from about 2000 to about 2500 g/mol. As used above, number averaged molecular weights (Mn) are based on the assumption of one olefin unsaturation per chain, as measured by ¹H NMR.

In a further embodiment, R₂ and R₃, as defined above, are independently a C₁-C₁₀ straight chained alklyene group. In another embodiment at least one of R₂, R₃ and R₈, as defined above, is an unsubstituted ethylene group. In one embodiment, a piperazine group can interrupt the R₂—NH—R₃ chain.

In some embodiments, the additive is a family of compounds represented by polypropylene succinic anhydride polyamines (PP-SA-PAMs) and poly(ethylene-co-propylene) succinic anhydride polyamines (EP-SA-PAMs). This class of antifouling additives are based on atactic or isotactic polypropylenes (PP) or poly(ethylene-co-propylene) (EP), which is functionalized with a polyamine (PAM) group in the chain end and a succinimide group as the linker. These functionalized polypropylenes or poly(ethylene-co-propylene) (e.g., with ethylene content of 10 to 90 wt %, or 20 to 50 wt %) can initially be prepared by metallocene-catalyzed polymerization of propylene or a mixture of ethylene and propylene, which are then terminated with a high vinyl group content in the chain end. The number-averaged molecular weight (M) of the PP or EP can be from about 300 to about 30,000 g/mol, or from about 500 to about 5000 g/mol, as determined by ¹H NMR spectroscopy and assuming one olefin unsaturation per chain. The vinyl-terminated atactic (i.e., randomly distributed) or isotactic polypropylenes (v-PP) or vinyl-terminated poly(ethylene-co-propylene) (v-EP) suitable for chemical functionalization and invention in the aforementioned refinery fouling mitigation can have a molecular weight (M) approximately from about 300 to about 30,000 g/mol, or from about 500 to 5000 g/mol as determined by ¹H NMR spectroscopy and assuming one olefin unsaturation per chain. The terminal olefin group can be a vinylidene group or an allylic vinyl group. In certain embodiments, the terminal olefin group is the allylic vinyl group, which differs from a vinylidene group. In this regard, the terminal allylic vinyl group rich PP or EP disclosed in co-pending application, U.S. application Ser. No. 12/143,663 can be used, which application is hereby incorporated by reference in its entirety.

In another embodiment, one or more of the R₁, R₇ and R₁₀ groups is independently selected from the group consisting of propylene polymers comprising propylene and less than 0.5 wt % comonomer, preferably 0 wt % comonomer, wherein the polymer has:
i) at least 93% allyl chain ends (preferably at least 95%, preferably at least 97%, preferably at least 98%);
ii) a number average molecular weight (Mn) of about 500 to about 20,000 g/mol, as measured by ¹H NMR, assuming one olefin unsaturation per chain (preferably 500 to 15,000, preferably 700 to 10,000, preferably 800 to 8,000 g/mol, preferably 900 to 7,000, preferably 1000 to 6,000, preferably 1000 to 5,000);
iii) an isobutyl chain end to allylic vinyl group ratio of 0.8:1 to 1.3:1.0;
iv) less than 1400 ppm aluminum, (preferably less than 1200 ppm, preferably less than 1000 ppm, preferably less than 500 ppm, preferably less than 100 ppm).

In another embodiment, one or more of the R₁, R₇ and R₁₀ groups is independently selected from the group consisting of propylene copolymers having an Mn of 300 to 30,000 g/mol as measured by 1H NMR and assuming one olefin unsaturation per chain (preferably 400 to 20,000, preferably 500 to 15,000, preferably 600 to 12,000, preferably 800 to 10,000, preferably 900 to 8,000, preferably 900 to 7,000 g/mol), comprising 10 to 90 mol % propylene (preferably 15 to 85 mol %, preferably 20 to 80 mol %, preferably 30 to 75 mol %, preferably 50 to 90 mol %) and 10 to 90 mol % (preferably 85 to 15 mol %, preferably 20 to 80 mol %, preferably 25 to 70 mol %, preferably 10 to 50 mol %) of one or more alpha-olefin comonomers (preferably ethylene, butene, hexene, or octene, preferably ethylene), wherein the polymer has at least X % allyl chain ends (relative to total unsaturations), where: 1) X=(−0.94 (mole % ethylene incorporated)+100{alternately 1.20 (−0.94 (mole % ethylene incorporated)+100), alternately 1.50(−0.94 (mole % ethylene incorporated)+100)}), when 10 to 60 mole % ethylene is present in the co-polymer, and 2) X=45 (alternately 50, alternately 60), when greater than 60 and less than 70 mole % ethylene is present in the co-polymer, and 3) X=(1.83*(mole % ethylene incorporated)−83, (alternately 1.20 [1.83*(mole % ethylene incorporated)−83], alternately 1.50 [1.83*(mole % ethylene incorporated)−83])), when 70 to 90 mole % ethylene is present in the copolymer. Alternately X is 80% or more, preferably 85% or more, preferably 90% or more, preferably 95% or more.

Alternatively, the polymer or copolymer has at least 80% isobutyl chain ends (based upon the sum of isobutyl and n-propyl saturated chain ends), preferably at least 85% isobutyl chain ends, preferably at least 90% isobutyl chain ends. Alternately, the polymer has an isobutyl chain end to allylic vinyl group ratio of 0.8:1 to 1.35:1.0, preferably 0.9:1 to 1.20:1.0, preferably 0.9:1.0 to 1.1:1.0.

In another embodiment, one or more of the R₁, R₇ and R₁₀ groups is independently selected from the group consisting of propylene polymers, comprising more than 90 mol % propylene (preferably 95 to 99 mol %, preferably 98 to 9 mol %) and less than 10 mol % ethylene (preferably 1 to 4 mol %, preferably 1 to 2 mol %), wherein the polymer has:

at least 93% allyl chain ends (preferably at least 95%, preferably at least 97%, preferably at least 98%);

a number average molecular weight (Mn) of about 400 to about 30,000 g/mol, as measured by ¹H NMR and assuming one olefin unsaturation per chain (preferably 500 to 20,000, preferably 600 to 15,000, preferably 700 to 10,000 g/mol, preferably 800 to 9,000, preferably 900 to 8,000, preferably 1000 to 6,000);

an isobutyl chain end to allylic vinyl group ratio of 0.8:1 to 1.35:1.0, and

less than 1400 ppm aluminum, (preferably less than 1200 ppm, preferably less than 1000 ppm, preferably less than 500 ppm, preferably less than 100 ppm).

In another embodiment, one or more of the R₁, R₇ and R₁₀ groups is independently selected from the group consisting of propylene polymers comprising:

at least 50 (preferably 60 to 90, preferably 70 to 90) mol % propylene and from 10 to 50 (preferably 10 to 40, preferably 10 to 30) mol % ethylene, wherein the polymer has:

at least 90% allyl chain ends (preferably at least 91%, preferably at least 93%, preferably at least 95%, preferably at least 98%);

an Mn of about 150 to about 20,000 g/mol, as measured by ¹H NMR and assuming one olefin unsaturation per chain (preferably 200 to 15,000, preferably 250 to 15,000, preferably 300 to 10,000, preferably 400 to 9,500, preferably 500 to 9,000, preferably 750 to 9,000); and

an isobutyl chain end to allylic vinyl group ratio of 0.8:1 to 1.3:1.0, wherein monomers having four or more carbon atoms are present at from 0 to 3 mol % (preferably at less than 1 mol %, preferably less than 0.5 mol %, preferably at 0 mol %).

In another embodiment, one or more of the R₁, R₇ and R₁₀ groups is independently selected from the group consisting of propylene polymers comprising:

at least 50 (preferably at least 60, preferably 70 to 99.5, preferably 80 to 99, preferably 90 to 98.5) mol % propylene, from 0.1 to 45 (preferably at least 35, preferably 0.5 to 30, preferably 1 to 20, preferably 1.5 to 10) mol % ethylene, and from 0.1 to 5 (preferably 0.5 to 3, preferably 0.5 to 1) mol % C₄ to C₁₂ olefin (such as butene, hexene or octene, preferably butene), wherein the polymer has:

at least 90% allyl chain ends (preferably at least 91%, preferably at least 93%, preferably at least 95%, preferably at least 98%);

a number average molecular weight (Mn) of about 150 to about 15,000 g/mol, as measured by ¹H NMR and assuming one olefin unsaturation per chain (preferably 200 to 12,000, preferably 250 to 10,000, preferably 300 to 10,000, preferably 400 to 9500, preferably 500 to 9,000, preferably 750 to 9,000); and

an isobutyl chain end to allylic vinyl group ratio of 0.8:1 to 1.35:1.0.

In another embodiment, one or more of the R₁, R₇ and R₁₀ groups is independently selected from the group consisting of propylene polymers comprising:

at least 50 (preferably at least 60, preferably 70 to 99.5, preferably 80 to 99, preferably 90 to 98.5) mol % propylene, from 0.1 to 45 (preferably at least 35, preferably 0.5 to 30, preferably 1 to 20, preferably 1.5 to 10) mol % ethylene, and from 0.1 to 5 (preferably 0.5 to 3, preferably 0.5 to 1) mol % diene (such as C₄ to C₁₂ alpha-omega dienes (such as butadiene, hexadiene, octadiene), norbornene, ethylidene norbornene, vinylnorbornene, norbornadiene, and dicyclopentadiene), wherein the polymer has:

at least 90% allyl chain ends (preferably at least 91%, preferably at least 93%, preferably at least 95%, preferably at least 98%);

a number average molecular weight (Mn) of about 150 to about 20,000 g/mol, as measured by ¹H NMR and assuming one olefin unsaturation per chain (preferably 200 to 15,000, preferably 250 to 12,000, preferably 300 to 10,000, preferably 400 to 9,500, preferably 500 to 9,000, preferably 750 to 9,000); and

an isobutyl chain end to allylic vinyl group ratio of 0.7:1 to 1.35:1.0.

Any of the propylene polymers prepared herein preferably have less than 1400 ppm aluminum, preferably less than 1000 ppm aluminum, preferably less than 500 ppm aluminum, preferably less than 100 ppm aluminum, preferably less than 50 ppm aluminum, preferably less than 20 ppm aluminum, preferably less than 5 ppm aluminum.

The terminal vinyl functionality in PP or EP can be maleated with excess maleic anhydride (1.0 to 5.0 equivalents) between 180 and 225° C. to give the corresponding polypropylenes or poly(ethylene-co-propylene)s with 0.8 to 2 succinic anhydride functionalities in the chain end. The maleation step can be a thermal process, or alternatively a catalyzed process with the use of suitable Lewis acid catalyst. Polyamines having varying chain length and number of amine functional group can be used to react with the succinic anhydride groups in the PP or EP chain end. In some embodiments, the polyamine has the structure

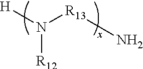

wherein R₁₂ is hydrogen or a C₁₋₁₀ branched or straight chained alkyl optionally substituted by one or more amine groups, R₁₃ is a C₁-C₁₀ branched or straight chained alkylene group, and x is an integer between 1 and 10 inclusive. Examples of the polyamines include, but are not limited to, ethyleneamine polymers such as diethylenetriamine (DETA), triethylenetetramine (TETA), tetraethylenepentamine (TEPA), pentaethylenehexamine (PEHA), hexaethyleneheptamine (HEHA) and higher molecular weight species, which can also contain a complex mixture of various linear, cyclic, and branched structures. The polyamine can provide overall functionalized polypropylene succinic anhydride polyamines (PP-SA-PAMs) or poly(ethylene-co-propylene) succinic anhydride polyamines (EP-SA-PAMs) composition, as schematically illustrated below for PP-SA-PAM.

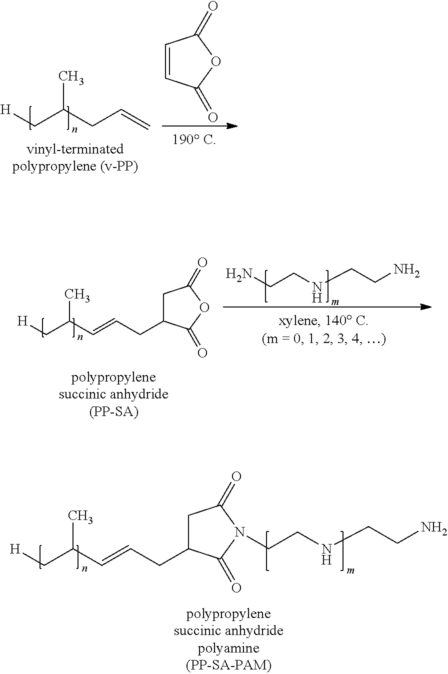

In the above reaction scheme, upon the reaction between the vinyl terminated polypropylene with maleic anhydride, the terminal double bond of the allylic vinyl group shifted, resulting in a bond between the original alpha carbon of the vinyl terminated PP with the succinic anhydride unit.

By selecting vinyl-terminated polypropylenes of different molecular weights and polyamines of different chain lengths and molecular composition (i.e., ethyleneamine polymers with a general formula of H₂N—(CH₂CH₂NH)ₘ—CH₂CH₂—NH₂ or propyleneamine polymers with a formula of H₂N—(CH₂CH₂CH₂NH)ₘ—CH₂CH₂CH₂—NH₂ where m=0, 1, 2, 3, 4, . . . ), the polypropylene-based additives can be molecularly designed to have different amount of basic nitrogen contents and hence varying degrees of dispersancy.

In a preferred embodiment, the synthesis processes described herein are continuous processes. As used herein the term continuous means a system that operates without interruption or cessation. For example a continuous process to produce a polymer would be one where the reactants are continually introduced into one or more reactors and polymer product is continually withdrawn.

A few representative examples illustrating the effects of molecular weight variation, type of polyamine used and stoichiometry to give PP-SA-PAM compositions different level of theoretical basic nitrogen content are shown in the following Table 1. The level of basic nitrogen can be controlled to provide a wide range of values from about 3 to 9% on a weight basis in the resulting dispersant additives.

TABLE 1
Different levels of theoretical basic nitrogen content as obtained
by varying the stoichiometry of PP-SA and polyamine.

| | NMR-averaged | | | Theoretical Basic |
|---|---|---|---|---|
| | MW of | | | Nitrogen |
| Entry | v-PP^{a} (g/mol) | PAM^{b} | PP-SA^{c}:PAM | Content (wt %) |
| 1 | 983.0 | TEPA | 2:1 | 3.0 |
| 2 | 983.0 | PEHA | 2:1 | 3.6 |
| 3 | 570.7 | TEPA | 2:1 | 4.7 |
| 4 | 570.7 | PEHA | 2:1 | 5.6 |
| 5 | 983.0 | TEPA | 1:1 | 5.6 |
| 6 | 983.0 | PEHA | 1:1 | 6.5 |
| 7 | 570.7 | TEPA | 1:1 | 8.3 |
| 8 | 570.7 | PEHA | 1:1 | 9.5 |

^{a}v-PP = vinyl-terminated polypropylene
^{b}PAM = polyamine, TEPA = tetraethylenepentamine, PEHA = pentaethylenehexamine
^{c}PP-SA = polypropylene succinic anhydride

In one embodiment, at least one of R₁, R₇ and R₁₀, as defined above, is polyisobutylene. In one embodiment, the additive is selected from

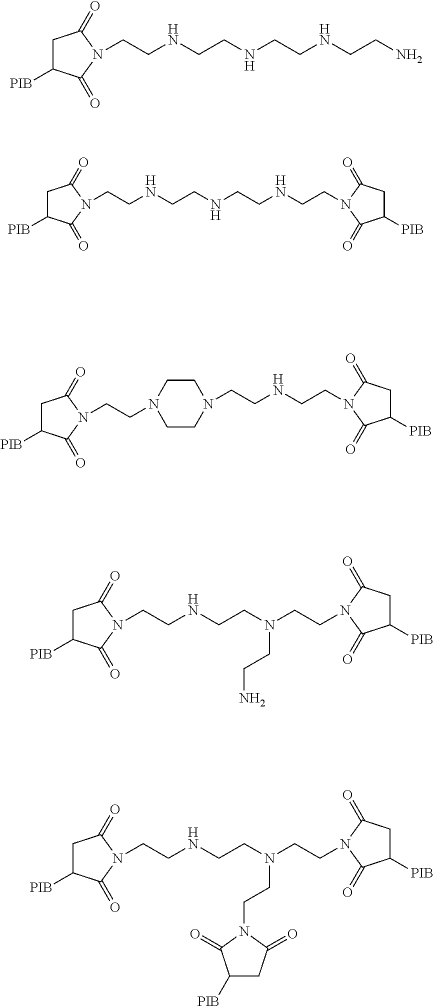

It will be understood by persons of ordinary skill in the art that, for example, the PIB group, as shown above, can be linked to the succinic anhydride group via an alkyl or alkenyl linking group. For example, when highly reactive polyisobutylene (HR-PIB) is employed having a terminal vinylidine terminal group, the following additives are provided, shown with a C₄ alkenyl linking group:

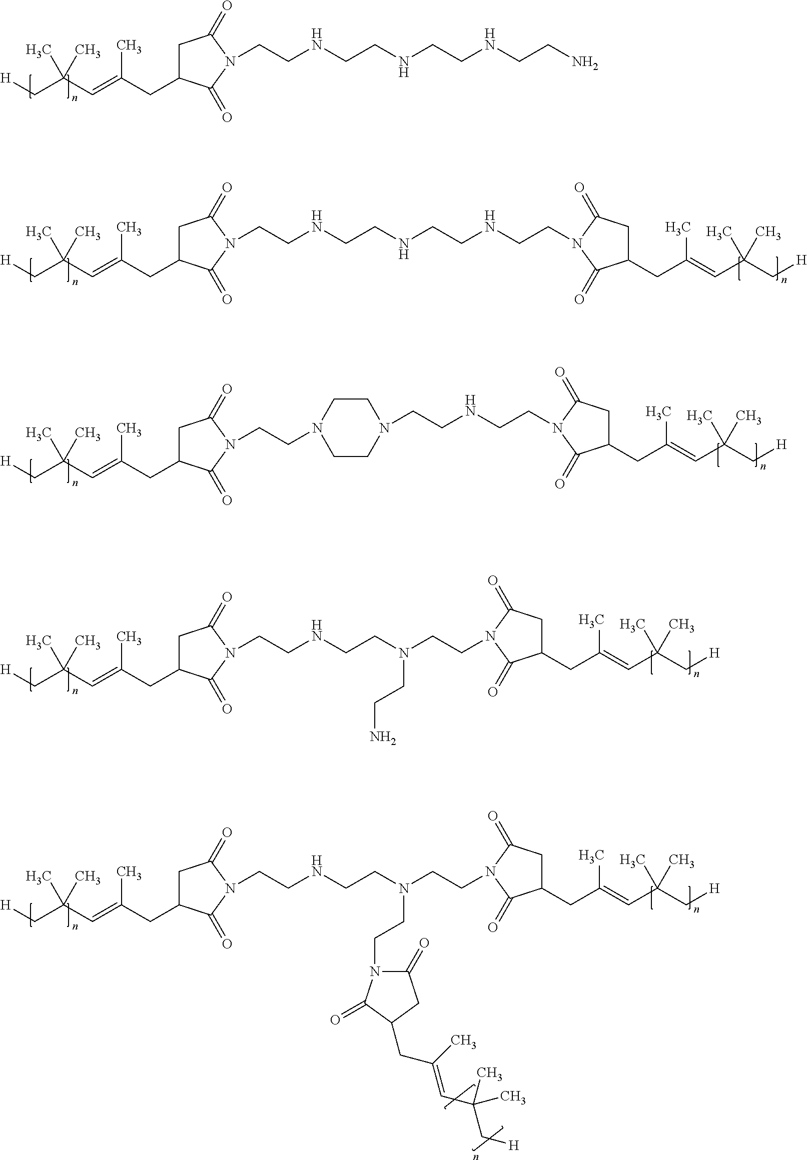

wherein n is from 1 to 200, or from about 1 to 100.

In one embodiment, any one of the above-described additives are associated or complexed with a boronating agent. In one embodiment, the boronating agent is selected from boric acid, an ortho-borate, or a meta-borate, for example, boric acid, trimethyl metaborate (trimethoxyboroxine), triethyl metaborate, tributyl metaborate, trimethyl borate, triethylborate, triisopropyl borate (triisopropoxyborane), tributyl borate (tributoxyborane) and tri-t-butyl borate.

Another aspect of the present invention provides a system for refining hydrocarbons that include at least one crude hydrocarbon refinery component, in which the crude hydrocarbon refinery component includes an additive selected from any one of the above-described additives. The crude hydrocarbon refining component can be selected from a heat exchanger, a furnace, a crude preheater, a coker preheater, a FCC slurry bottom, a debutanizer exchanger, a debutanizer tower, a feed/effluent exchanger, a furnace air preheater, a flare compressor component, a steam cracker, a steam reformer, a distillation column, a fractionation column, a scrubber, a reactor, a liquid-jacketed tank, a pipestill, a coker, and a visbreaker. In one preferred embodiment, the crude hydrocarbon refining component is a heat exchanger (e.g. a crude pre-heat train heat exchanger).

Another aspect of the present invention provides a composition for reducing fouling that includes at least one of any of the above-described additives, and a boronating agent. The boronating agent is selected from boric acid, an ortho-borate, or a meta-borate, for example, boric acid, trimethyl metaborate (trimethoxyboroxine), triethyl metaborate, tributyl metaborate, trimethyl borate, triethylborate, triisopropyl borate (triisopropoxyborane), tributyl borate (tributoxyborane) and tri-t-butyl borate.

Exemplary further embodiments of the present invention are provided below for illustrative purposes, and not for purposes of limitation.

### Applications for the Polyalkyl Succinic Anyhydride Polyamine Additives

The additives of the present invention are generally soluble in a typical hydrocarbon refinery stream and can thus be added directly to the process stream, alone or in combination with other additives that either reduce fouling or improve some other process parameter.

The additives can be introduced, for example, upstream from the particular crude hydrocarbon refinery component(s) (e.g. a heat exchanger) in which it is desired to prevent fouling (e.g. particulate induced fouling). Alternatively, the additive can be added to the crude oil prior to being introduced to the refining process, or at the very beginning of the refining process.

It is noted that water can have a negative impact on boron-containing additives. Accordingly, it is advisable to add boron-containing additives at process locations that have a minimal amount of water.

While not limited thereto, the additives of the present invention are particularly suitable in reducing or preventing particulate-induced fouling. Thus one aspect of the present invention provides a method of reducing and/or preventing, in particular, particulate-induced fouling that includes adding at least one additive of the present invention to a process stream that is known, or believed to contribute to, particulate-induced fouling. To facilitate determination of proper injection points, measurements can be taken to ascertain the particulate level in the process stream. Thus, one embodiment of the present invention includes identifying particular areas of a refining process that have relatively high particulate levels, and adding any one of the additives of the present invention in close proximity to these areas (e.g., just upstream to the area identified as having high particulate levels).

In one embodiment of the present invention, a method to reduce fouling is provided comprising adding any one of the above-mentioned additives to a crude hydrocarbon refinery component that is in fluid communication with a process stream that contains, at least 50 wppm of particulates, including organic and inorganic particulates. In another embodiment of the present invention, a method to reduce fouling is provided comprising adding any one of the above-mentioned additives to a crude hydrocarbon refinery component that is in fluid communication with a process stream that contains at least 250 wppm (or 1000 wppm, or 10,000 wppm) of particulates, including organic and inorganic particulates, as defined above.

In one embodiment of the present invention, the additives of the present invention are added to selected crude oil process streams known to contain, or possibly contain, problematic amounts of organic or inorganic particulate matter (e.g. 1-10,000 wppm), such as inorganic salts. Accordingly, the additives of the present invention can be introduced far upstream, where the stream is relatively unrefined (e.g. the refinery crude pre-heat train). The additives can be also added, for example, after the desalter to counteract the effects of incomplete salt removal or to the bottoms exit stream from the fractionation column to counteract the high temperatures that are conducive to fouling.

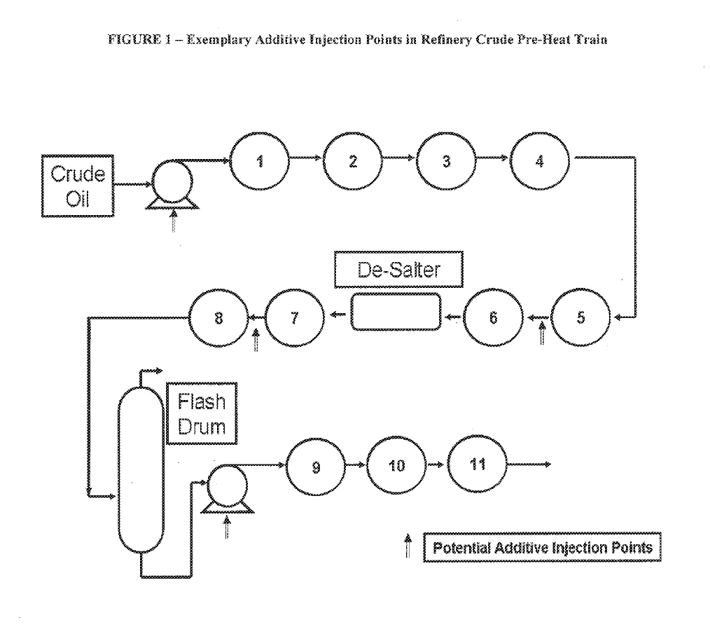

FIG. 1 demonstrates possible additive injection points within the refinery crude pre-heat train for the additives of the present invention, wherein the numbered circles represent heat exchangers. As shown in FIG. 1, the additives can be introduced in crude storage tanks and at several locations in the preheat train. This includes at the crude charge pump (at the very beginning of the crude pre-heat train), and/or before and after the desalter, and/or to the bottoms stream from a flash drum.

The total amount of additive to be added to the process stream can be determined by a person of ordinary skill in the art. In one embodiment, up to about 1000 wppm of additive is added to the process stream. For example, the additive can be added such that its concentration, upon addition, is about 50 ppm, 250 ppm or 500 ppm. More or less additive can be added depending on, for example, the amount of particulate in the stream, the ΔT associated with the particular process and the degree of fouling reduction desired in view of the cost of the additive.

The additives of the present invention can be added in a solid (e.g. powder or granules) or liquid form directly to the process stream. As mentioned above, the additives can be added alone, or combined with other components to form a composition for reducing fouling (e.g. particulate-induced fouling). Any suitable technique can be used for adding the additive to the process stream, as known by a person of ordinary skill in the art in view of the process to which it is employed. As a non-limiting example, the additives can be introduced via injection that allows for sufficient mixing of the additive and the process stream.

### Obtaining the Additives of the Present Invention

Certain (non-borate) additives of the present invention can be obtained from commercial sources. For example, certain additives of the present invention can also be obtained from Chevron Oronite Company LLC (San Ramon, Calif.), including Oronite™ OLOA 11000. These products are described as useful as additives for gasoline and natural gas engines as well as additives for gear oils and hydraulic fluids. Additive of the present application can also be obtained from Infineum Co. (Oxfordshire, UK and Linden, N.J.), including Infineum™ C-9230.

In certain embodiments, the alkyl group on additives of the present invention is polyisobutylene (R₁, R₇, and/or R₁₀ as defined above). The polyisobutylene (PIB) can be normal PIB (i.e., “conventional”) and/or Highly Reactive PIB (HRPIB). HRPIB is generally characterized has having a vinylidene double bond content from about 1% to about 100%, and can be obtained from, for example, BASF (Ludwigshafen, Germany).

### Obtaining Boronating Agents of the Present Application

Boronating agents for use in the present invention can be obtained by persons of ordinary skill in the art from commercial vendors. Non-limiting examples of vendors include products available from ExxonMobil Chemical Co. (Houston, Tex.) under the “Mobilad”™ brand, particularly Mobilad™ C-700 and Mobilad™ C-701.

Boronating agents of the present invention can also be synthesized by persons of ordinary skill in the art, in view of, for example, the exemplary reaction schemes disclosed in U.S. Pat. Nos. 5,804,667, 5,936,041, 5,026,495, 5,788,722 and 6,030,930, each of which is hereby incorporated by reference in their entirety.

### Preparing Boron-Modified Additives of the Present Application

The boron-modified additives of the present invention, i.e. an additive complexed or in association with a boronating agent, can be prepared by introducing (e.g. mixing) a non-borate additive with a boronating agent. Preferably, the mixture is heated (e.g. heated up to about 80° C.) for about 1-2 hours to obtain the boron-complexed additive.

Alternatively boron-modified additives of the present invention can be directly purchased from commercial vendors. For example, various boron-containing succinic acid ester additives are available from the Infineum Co. (Oxfordshire, UK and Linden, N.J.), including Infineum™ C-9230. Boron-containing additives can also be obtained from Afton Chemical Co. (Richmond, Va.) such as Afton Hitec™ 643D.

An example of a boron-modified additive of the present invention, shown solely for illustrative purposes, and not for purposes of limitation, is shown below:

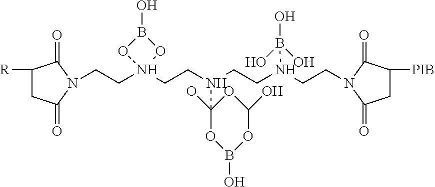

One embodiment of the present invention provides boron-modified additives with a particularly high boron content (e.g., above 1 wt %, 2 wt % or 5 wt % boron). To achieve these relatively high amounts of boron, it is possible to introduce a commercially available boron-containing additive with a boronating agent to further increase the boron content of the additive.

While not being bound by any particular theory, it is believed that the polyamine group and the borate group complex together to form a strong polar network that significantly contributes to the further increase the anti-fouling effects, as compared to the additive without the borate group.

In one embodiment of the present invention, the boron-modified additive contains at least 1 wt %, or at least 2 wt %, or at least 5 wt % boron. Weight ratios of nitrogen:boron can range from about 1:5 to about 5:1, more preferably from about 1:2 to 2:1.

### Compositions for Reducing Fouling

The additives of the present invention can be used in compositions that prevent fouling, including particulate-induced fouling. In addition to the additives of the present invention, the compositions can further contain a hydrophobic oil solubilizer for the additive and/or a dispersant for the additive.

Suitable solubilizers can include, for example, surfactants, carboxylic acid solubilizers, such as the nitrogen-containing phosphorous-free carboxylic solubilizers disclosed in U.S. Pat. No. 4,368,133, hereby incorporated by reference in its entirety.

Also as disclosed in U.S. Pat. No. 4,368,133, hereby incorporated by reference, surfactants that can be included in compositions of the present invention can include, for example, any one of a cationic, anionic, nonionic or amphoteric type of surfactant. See, for example, McCutcheon's “Detergents and Emulsifiers”, 1978, North American Edition, published by McCutcheon's Division, MC Publishing Corporation, Glen Rock, N.J., U.S.A., including pages 17-33, which is hereby incorporated by reference in its entirety.

The compositions of the present invention can further include, for example, viscosity index improvers, anti-foamants, antiwear agents, demulsifiers, anti-oxidants, and other corrosion inhibitors.

Furthermore, the additives of the present invention can be added with other compatible components that address other problems that can present themselves in an oil refining process known to one of ordinary skill in the art.

### EXAMPLES

The present invention is further described by means of the examples, presented below. The use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification. The invention is therefore to be limited only by the terms of the appended claims along with the full scope of equivalents to which the claims are entitled.

### Example 1

### Synthesis of Various Additives of the Present Invention

Several commercially available PIBSA-PAM-Ester products or boron-containing PIBSA-PAM-Ester products made from either conventional PIB or Highly Reactive-PIB (HR-PIB) were blended with organic borates at elevated temperatures to form a series of new products with high boron content in the following manner:

### Example A

### Synthesis of Additive A

37.5 grams of a commercial, boron-containing succinimide/succinic acid ester dispersant (Infineum C-9230 with 1.3 wt % boron and 1.2 wt % nitrogen commercially available from Infineum Co.) were mixed with 12.5 grams of an organic boron additive [Mobilad C-700, 5.6 wt % boron, commercially available from ExxonMobil Chemical Co. (Houston, Tex.)] and the viscous mixture was heated to 80° C. for about one hour. The resulting final adduct upon cooling is a light brownish liquid [elemental analysis, boron: 2.56 wt %, nitrogen: 0.83 wt %].

### Example B

### Synthesis of Additive B

A commercial boron-free, succinimide/succinic acid ester dispersant, (Infineum 9268, 1.2 wt % nitrogen) was used as an anti-fouling agent.

### Example C

### Synthesis of Additive C

37.5 grams of a commercial, boron-containing succinimide/succinic acid ester dispersant [Afton Hitec 643D with 0.8 wt % boron and 1.6 wt % nitrogen, commercially available from Afton Chemical Co. (Richmond, Va.)] were mixed with 12.5 grams of an organic boron additive [Mobilad C-700] and the viscous mixture was heated to 80° C. for about one hour. The resulting final adduct upon cooling is a light brownish liquid [elemental analysis, boron: 2.36 wt %, nitrogen: 1.1 wt %].

### Example D

### Synthesis of Additive D

37.5 grams of a commercial succinimide/succinic acid ester dispersant [Oronite OLOA 11000 with 3.2 wt % nitrogen, commercially available from Chevron Oronite Corp. (San Ramon, Calif.)] were mixed with 12.5 grams of an organic boron additive [Mobilad C-700] and the viscous mixture was heated to 80° C. for about 1.5 hour. The resulting final adduct upon cooling is a dark brownish, very viscous liquid [elemental analysis, boron: 1.2 wt %].

### Example E

### Synthesis of Additive E

25 grams of a commercial succinimide/succinic acid ester dispersant [Oronite OLOA 11000 with 3.2 wt % nitrogen] were mixed with 25 grams of an organic boron additive [Mobilad C-700] and the viscous mixture was heated to 80° C. for about 1.5 hour. The resulting final adduct upon cooling is a dark brownish, very viscous liquid [elemental analysis, boron: 3.5 wt %].

### Example F

### Synthesis of Additive F

A commercial boron-free, succinimide/succinic acid ester dispersant, (Afton Hitec 638, 2.0 wt % nitrogen) was used as the anti-fouling agent.

### Example G

### Synthesis of Additive G

25 grams of a commercial, boron-containing succinimide/succinic acid ester dispersant [Infineum C-9230 with 1.3 wt % boron and 1.2 wt % nitrogen] were mixed with 25 grams of an organic boron additive [Mobilad C-701, 2.9 wt % boron] and the viscous mixture was heated to 80° C. for about one hour. The resulting final adduct upon cooling is a dark brownish liquid [elemental analysis, boron: 2.3 wt %, nitrogen: 0.3 wt %].

### Example H

### Synthesis of Additive H

A commercial dispersant modified organic borate additives, succinimide/succinic acid ester dispersant, (Infineum C9230, 1.24 wt % nitrogen, and 1.3 wt % boron) was used as an anti-fouling agent.

### Example I

### Synthesis of Additive I

20 grams of a commercial succinimide/succinic acid ester dispersant [Oronite OLOA 11000 with 3.2 wt % nitrogen] were mixed with 30 grams of an organic boron additive [Mobilad C-700, 5.6 wt % boron] and the viscous mixture was heated to 80° C. for about 1.5 hour. The resulting final adduct upon cooling is a dark brownish, very viscous liquid.

### Example J

### Synthesis of Additive J

30 grams of a commercial succinimide/succinic acid ester dispersant [Oronite OLOA 11000 with 3.2 wt % nitrogen] were mixed with 20 grams of an organic boron additive [Mobilad C-700, 5.6 wt % boron] and the viscous mixture was heated to 80° C. for about 1.5 hour. The resulting final adduct upon cooling is a dark brownish, very viscous liquid.

### Example K

### Synthesis of Additive K

20 grams of a commercial succinimide/succinic acid ester dispersant [Oronite OLOA 11000 with 3.2 wt % nitrogen] were mixed with 10 grams technical grade xylene and 30 grams of an organic boron additive [Mobilad C-700, 5.6 wt % boron] and the viscous mixture was heated to 80° C. for about 1.5 hour. The resulting final adduct upon cooling is a dark brownish, very viscous liquid.

### Example L

### Synthesis of Additive L

25 grams of a commercial, boron-containing succinimide/succinic acid ester dispersant [Infineum C-9230 with 1.3 wt % boron and 1.2 wt % nitrogen] were mixed with 25 grams of an organic boron additive [Mobilad C-700] and the viscous mixture was heated to 80° C. for about one hour. The resulting final adduct upon cooling is a light brownish liquid [elemental analysis, boron: 3.5 wt %, nitrogen: 0.4 wt %].

### Example M

### Synthesis of Additive M

37.5 grams of a commercial, boron-containing succinimide/succinic acid ester dispersant [Mobilad C-200 with 1.8 wt % boron and 1.6 wt % nitrogen] were mixed with 12.5 grams of an organic boron additive [Mobilad C-700] and the viscous mixture was heated to 80° C. for about one hour. The resulting final adduct upon cooling is a dark brownish liquid [elemental analysis, boron: 3 wt %, nitrogen: 1.2 wt %].

### Example N

### Synthesis of Additive N

25 grams of a commercial, boron-containing succinimide/succinic acid ester dispersant [Oronite OLOA 11000 with 3.2 wt % nitrogen] were mixed with 25 grams of an organic boron additive [Mobilad C-701, 2.9 wt % boron] and the viscous mixture was heated to 80° C. for about 1.5 hour. The resulting final adduct upon cooling is a dark brownish, very viscous liquid [elemental analysis, boron: 2.1 wt %, nitrogen: 2.2 wt %].

### Example O

### Synthesis of Additive PP-SA-PAM and EP-SA-PAM

(a) Maleation of Vinyl-Terminated Polypropylene or Poly(Ethylene-Co-Propylene) to Obtain Polypropylene Succinic Anhydride (PP-SA) or Poly(Ethylene-Co-Propylene) Succinic Anhydride (EP-SA)

A mixture of vinyl-terminated polypropylene (NMR averaged molecular weight 570.73, assuming one olefin unsaturation per chain) (10.00 g, 17.52 mmol) and maleic anhydride (8.59 g, 87.60 mmol, 5.0 equiv.) was heated at 190° C. (oil bath) under a nitrogen atmosphere for 22 hr. The mixture was allowed to cool to room temperature, diluted with hexanes, filtered, and concentrated to give a brown oil. This crude product was then heated at 110° C. under high vacuum to remove excess maleic anhydride to provide a brown oil (10.5 g). The structure and purity of the crude product was established by ¹H and ¹³C NMR (CDCl₃, 400 and 100 MHz, respectively), which confirmed complete conversion of the terminal vinyl group to the corresponding succinic anhydride group. This product has a succinic anhydride content of 1.457 mol/g based on elemental analysis.

A mixture of vinyl-terminated polypropylene (NMR averaged molecular weight 944.67, assuming one olefin unsaturation per chain) (5.40 g, 5.72 mmol) and maleic anhydride (1.68 g, 17.13 mmol, 3.0 equiv.) was heated at 190° C. (oil bath) under a nitrogen atmosphere for 24 hr. The mixture was allowed to cool to room temperature, diluted with hexanes, filtered, and concentrated to give a dark brown oil. This crude product was then heated at 110° C. under high vacuum to remove excess maleic anhydride to provide a dark brown oil (5.3 g). This product has a succinic anhydride content of 1.198 mol/g based on elemental analysis.

A mixture of vinyl-terminated polypropylene (NMR averaged molecular weight 2331.69, assuming one olefin unsaturation per chain) (10.00 g, 4.29 mmol) and maleic anhydride (2.10 g, 21.42 mmol, 5.0 equiv.) was heated at 190° C. (oil bath) under a nitrogen atmosphere for 96 hr. The mixture was allowed to cool to room temperature, diluted with hexanes, filtered, and concentrated to give a dark brown oil. This crude product was then heated at 110° C. under high vacuum to remove excess maleic anhydride to provide a dark brown oil (8.6 g). This product has a succinic anhydride content of 0.481 mol/g based on elemental analysis.

A mixture of vinyl-terminated polypropylene (NMR averaged molecular weight 2331.69, assuming one olefin unsaturation per chain) (20.00 g, 8.58 mmol) and maleic anhydride (4.21 g, 42.93 mmol, 5.0 equiv.) was heated at 190° C. (oil bath) under a nitrogen atmosphere for 106 hr. The mixture was allowed to cool to room temperature, diluted with hexanes, filtered, and concentrated to give a dark brown oil. This crude product was then heated at 110° C. under high vacuum to remove excess maleic anhydride to provide a dark brown oil (21.5 g). This product has a succinic anhydride content of 0.723 mol/g based on elemental analysis.

A mixture of vinyl-terminated poly(ethylene-co-propylene) (NMR averaged molecular weight 828, assuming one olefin unsaturation per chain) (3.00 g, 3.62 mmol) and maleic anhydride (1.065 g, 10.86 mmol, 3.0 equiv.) was heated at 190° C. (oil bath) under a nitrogen atmosphere for 24 hr. Additional maleic anhydride (1.065 g, 10.86 mmol, 3.0 equiv.) was added and the mixture was heated at 190° C. for an additional 24 hr. After this period additional maleic anhydride (1.78 g, 18.15 mmol, 5.0 equiv) was added and the mixture was heated at 190° C. for an additional 48 hr. The mixture was allowed to cool to room temperature, diluted with hexanes, filtered, and concentrated to give a dark brown gel-like product. This crude product was then heated at 110° C. under high vacuum to remove excess maleic anhydride to provide a dark brown gel-like product (2.75 g). This product has a succinic anhydride content of 2.22 mol/g based on elemental analysis.

(b) Synthesis of Polypropylene Succinic Anhydride Polyamine (PP-SA-PAM) and Poly(Ethylene-Co-Propylene) Succinic Anhydride Polyamine (EP-SA-PAM):

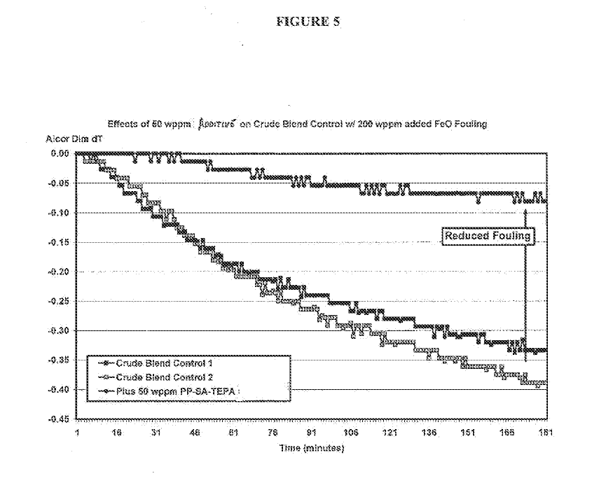

A mixture of polypropylene succinic anhydride (3.89 g, 5.67 mmol, 1.45 equiv.), tetraethylenepentamine (0.738 g, 3.90 mmol, 1.0 equiv.) and xylene (45 ml) was heated at reflux (oil bath temperature 170° C.) under a nitrogen atmosphere for 24 hr. A Dean-Stark trap was used to collect any water formed in the condensation reaction. After the reaction was completed, the mixture was allowed to cool to room temperature, and excess xylene was removed initially on a rotary evaporator followed by heating under high vacuum to afford a viscous brown oil (3.98 g) as crude product. This product has a total nitrogen content of 5.83 wt. %. The structure and purity of the crude product was established by ¹H and ¹³C NMR (CDCl₃, 400 and 100 MHz, respectively), which confirmed complete conversion of the succinic anhydride group to the corresponding succinimide linkage. The anti-fouling effects of this additive is shown in FIG. 5 of this application.

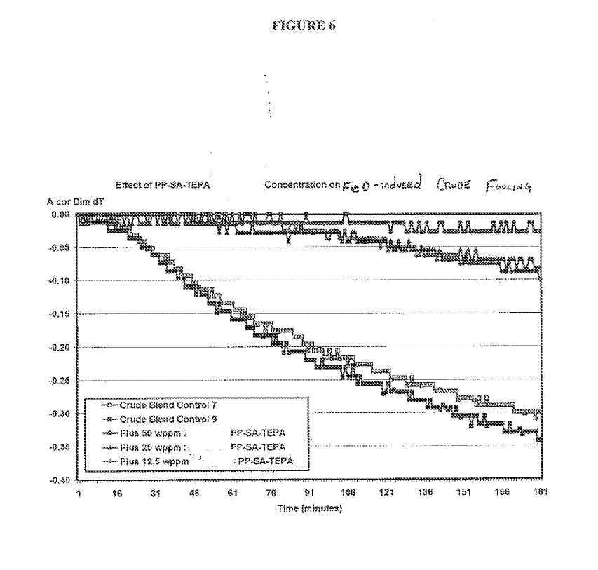

A mixture of polypropylene succinic anhydride (10.62 g, 12.72 mmol, 1.22 equiv.), tetraethylenepentamine (1.97 g, 10.41 mmol, 1.0 equiv.) and xylene (45 ml) was heated at reflux (oil bath temperature 170° C.) under a nitrogen atmosphere for 36 hr. A Dean-Stark trap was used to collect any water formed in the condensation reaction. After the reaction was completed, the mixture was allowed to cool to room temperature, and excess xylene was removed initially on a rotary evaporator followed by heating under high vacuum to afford a viscous dark brown oil (11.5 g) as crude product. This product has a total nitrogen content of 5.70 wt. %. The structure and purity of the crude product was established by ¹H and ¹³C NMR (CDCl₃, 400 and 100 MHz, respectively), which confirmed complete conversion of the succinic anhydride group to the corresponding succinimide linkage. The anti-fouling effects of this additive is shown in FIG. 6 of this application.

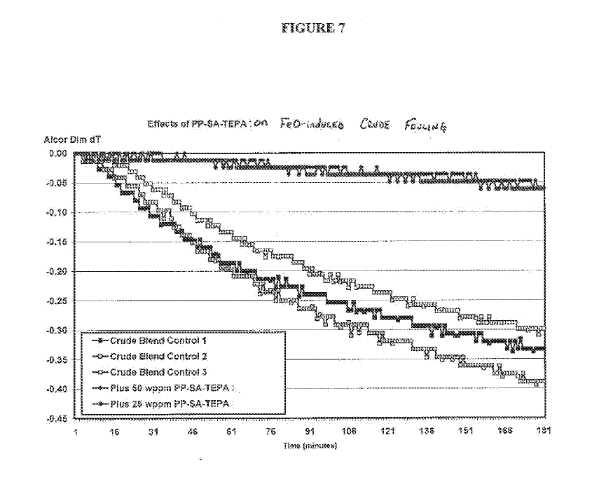

A mixture of polypropylene succinic anhydride (3.71 g, 1.78 mmol, 1.22 equiv.), tetraethylenepentamine (0.277 g, 1.46 mmol, 1.0 equiv.) and xylene (45 ml) was heated at reflux (oil bath temperature 170° C.) under a nitrogen atmosphere for 24 hr. A Dean-Stark trap was used to collect any water formed in the condensation reaction. After the reaction was completed, the mixture was allowed to cool to room temperature, and excess xylene was removed initially on a rotary evaporator followed by heating under high vacuum to afford a viscous brown oil (3.5 g) as crude product. This product has a total nitrogen content of 2.33 wt. %. The structure and purity of the crude product was established by ¹H and ¹³C NMR (CDCl₃, 400 and 100 MHz, respectively), which confirmed complete conversion of the succinic anhydride group to the corresponding succinimide linkage. The anti-fouling effects of this additive is shown in FIG. 7 of this application.

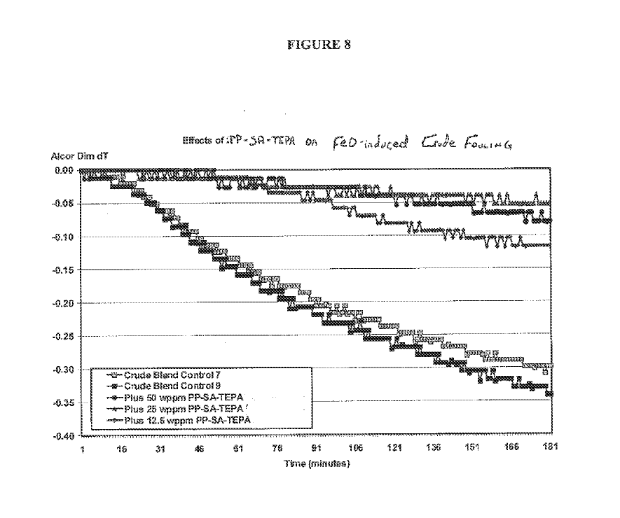

A mixture of polypropylene succinic anhydride (4.00 g, 2.892 mmol, 1.22 equiv.), tetraethylenepentamine (0.45 g, 2.37 mmol, 1.0 equiv.) and xylene (50 ml) was heated at reflux (oil bath temperature 175° C.) under a nitrogen atmosphere for 72 hr. A Dean-Stark trap was used to collect any water formed in the condensation reaction. After the reaction was completed, the mixture was allowed to cool to room temperature, and excess xylene was removed initially on a rotary evaporator followed by heating under high vacuum to afford a viscous brown oil (3.8 g) as crude product. This product has a total nitrogen content of 3.38 wt. %. The structure and purity of the crude product was established by ¹H and ¹³C NMR (CDCl₃, 400 and 100 MHz, respectively), which confirmed complete conversion of the succinic anhydride group to the corresponding succinimide linkage. The anti-fouling effects of this additive is shown in FIG. 8 of this application.

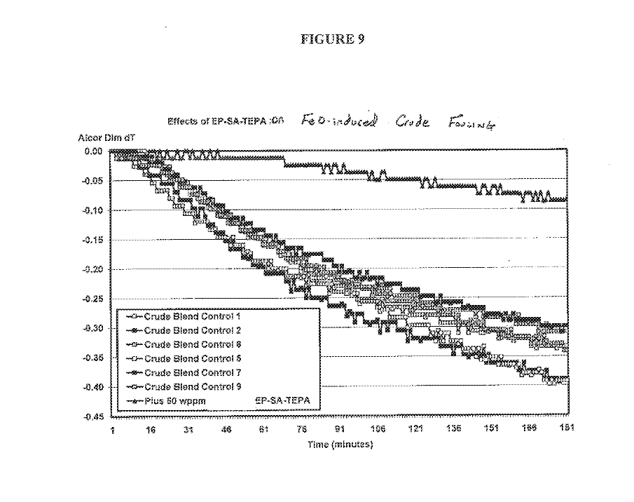

A mixture of poly(ethylene-co-propylene) succinic anhydride (2.60 g, 5.77 mmol, 1.22 equiv.), tetraethylenepentamine (0.895 g, 4.73 mmol, 1.0 equiv.) and xylene (45 ml) was heated at reflux (oil bath temperature 170° C.) under a nitrogen atmosphere for 52 hr. A Dean-Stark trap was used to collect any water formed in the condensation reaction. After the reaction was completed, the mixture was allowed to cool to room temperature, and excess xylene was removed initially on a rotary evaporator followed by heating under high vacuum to afford a dark brown oily solid (2.8 g) as crude product. This product has a total nitrogen content of 8.42 wt. %. The structure and purity of the crude product was established by ¹H and ¹³C NMR (CDCl₃, 400 and 100 MHz, respectively), which confirmed complete conversion of the succinic anhydride group to the corresponding succinimide linkage. The anti-fouling effects of this additive is shown in FIG. 9 of this application.

### Example 2

### Fouling Reduction Measured in the Alcor HLPS (Hot Liquid Process Simulator)

FIG. 2 depicts an Alcor HLPS (Hot Liquid Process Simulator) testing apparatus used to measure what the impact the addition of particulates to a crude oil has on fouling and what impact the addition of an additive of the present invention has on the reduction and mitigation of fouling. The testing arrangement includes a reservoir 10 containing a feed supply of crude oil. The feed supply of crude oil can contain a base crude oil containing a whole crude or a blended crude containing two or more crude oils. The feed supply is heated to a temperature of approximately 150° C./302° F. and then fed into a shell 11 containing a vertically oriented heated rod 12. The heated rod 12 is formed from carbon-steel (1018). The heated rod 12 simulates a tube in a heat exchanger. The heated rod 12 is electrically heated to a surface temperature of 370° C./698° F. or 400° C./752° F. and maintained at such temperature during the trial. The feed supply is pumped across the heated rod 12 at a flow rate of approximately 3.0 mL/minute. The spent feed supply is collected in the top section of the reservoir 10. The spent feed supply is separated from the untreated feed supply oil by a sealed piston, thereby allowing for once-through operation. The system is pressurized with nitrogen (400-500 psig) to ensure gases remain dissolved in the oil during the test. Thermocouple readings are recorded for the bulk fluid inlet and outlet temperatures and for surface of the rod 12.

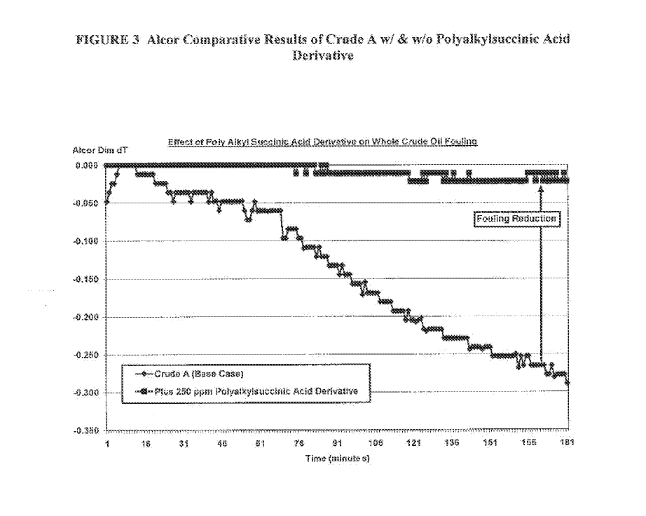

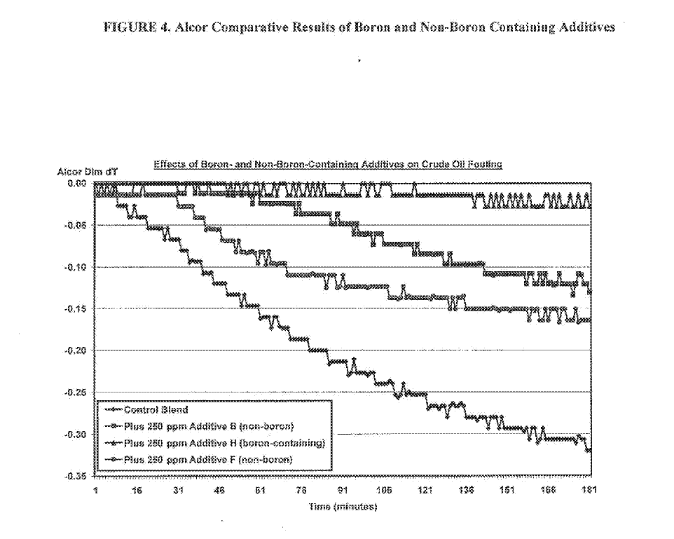

During the constant surface temperature testing, foulant deposits and builds up on the heated surface. The foulant deposits are thermally degraded to coke. The coke deposits cause an insulating effect that reduces the efficiency and/or ability of the surface to heat the oil passing over it. The resulting reduction in outlet bulk fluid temperature continues over time as fouling continues. This reduction in temperature is referred to as the outlet liquid ΔT or ΔT and can be dependent on the type of crude oil/blend, testing conditions and/or other effects, such as the presence of salts, sediment or other fouling promoting materials. A standard Alcor fouling test is carried out for 180 minutes. The total fouling, as measured by the total reduction in outlet liquid temperature over time, is plotted on the y-axis of FIG. 3 and FIG. 4 and is the observed outlet temperature (Tₒᵤₜₗₑₜ) minus the maximum observed outlet T_{outlet max} (presumably achieved in the absence of any fouling).

FIG. 3 illustrates the impact of fouling of a refinery component over 180 minutes. Two streams were tested in the Alcor unit: a crude oil control without an additive, and the same stream with 250 ppm of Infineum C9268, a commercially available polyisobutyl succinic acid-polyamine ester. As FIG. 3 demonstrates, the reduction in the outlet temperature over time (due to fouling) is less for the process stream containing 250 ppm of additive as compared to the crude oil control without the additive. This indicates that Infineum C9268 is effective at reducing fouling of a heat exchanger.

FIG. 4 demonstrates the results of the same test, except that two non-boron additives, one boron-containing additive, and a control blend (no additive) were tested in the Alcor unit to determine, inter alia, the effect that boron has on fouling reduction. More particularly the control stream was modified by adding, in three separate formulations, 250 ppm of additive B, 250 ppm of additive H and 250 ppm of Additive F. As FIG. 4 indicates, all three additives were effective at reducing fouling, and the boron-containing additive (Additive H) reduced fouling to the greatest extent.

FIG. 5 demonstrates the effects of fouling of control crude oil blend samples (Crude Blend Control 1 and Crude Blend Control 2, both containing 200 wppm added iron oxide particles) and a crude oil blend sample treated with 50 wppm of a PP-SA-TEPA additive having a total nitrogen content of 5.83 wt % and a polypropylene chain of about 571 g/mol, as measured in the Alcor HLPS apparatus depicted in FIG. 2. The comparative results of FIG. 5 clearly show that adding the PP-SA-TEPA additive was effective for reducing fouling.

FIG. 6 demonstrates the effects of fouling of control crude oil blend samples (Crude Blend Control 7 and Crude Blend Control 9, both containing 200 wppm added iron oxide particles) and a crude oil blend sample treated with 50, 25 and 12.5 wppm of a PP-SA-TEPA additive having a total nitrogen content of 5.70 wt % and a polypropylene chain of about 945 g/mol, as measured in the Alcor HLPS apparatus depicted in FIG. 2. The comparative results of FIG. 6 clearly show that adding the PP-SA-TEPA additive was effective for reducing fouling.

FIG. 7 demonstrates the effects of fouling of control crude oil blend samples (Crude Blend Control 1, Crude Blend Control 2 and Crude Blend Control 3, each containing 200 wppm added iron oxide particles) and a crude oil blend sample treated with 50 and 25 wppm of a PP-SA-TEPA additive having a total nitrogen content of 2.33 wt % and a polypropylene chain of about 2,332 g/mol, as measured in the Alcor HLPS apparatus depicted in FIG. 2. The comparative results of FIG. 7 clearly show that adding the PP-SA-TEPA additive was effective for reducing fouling.

FIG. 8 demonstrates the effects of fouling of control crude oil blend samples (Crude Blend Control 7 and Crude Blend Control 9, both containing 200 wppm added iron oxide particles) and a crude oil blend sample treated with 50, 25 and 12.5 wppm of a PP-SA-TEPA additive having a total nitrogen content of 3.38 wt % and a polypropylene chain of about 2,332 g/mol, as measured in the Alcor HLPS apparatus depicted in FIG. 2. The comparative results of FIG. 8 clearly show that adding the PP-SA-TEPA additive was effective for reducing fouling.

FIG. 9 demonstrates the effects of fouling of control crude oil blend samples (Crude Blend Control 1, Crude Blend Control 2, Crude Blend Control 5, Crude Blend Control 7, Crude Blend Control 8 and Crude Blend Control 9, each containing 200 wppm added iron oxide particles) and a crude oil blend sample treated with 50 wppm of a EP-SA-TEPA additive having a total nitrogen content of 8.42 wt % and a poly(ethylene-co-propylene) chain of about 828 g/mol, as measured in the Alcor HLPS apparatus depicted in FIG. 2. The comparative results of FIG. 9 clearly show that adding the EP-SA-TEPA additive was effective for reducing fouling.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, priority documents, publications, product descriptions, and protocols are cited throughout this application, the disclosures of each of which is incorporated herein by reference in its entirety for all purposes.

1.-17. (canceled)

18. A composition for reducing fouling, comprising: 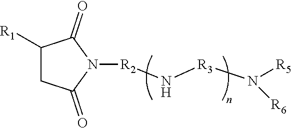
wherein R₁ is a branched or straight-chained C₁₀-C₈₀₀ alkyl or alkenyl group;
n is an integer from 1 to 10;
R₂ and R₃ are independently a C₁-C₁₀ branched or straight chained alkylene group;
R₅ and R₆ are H or R₅ and R₆ together along with the N atom bound thereto form the group: 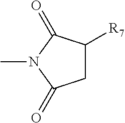
wherein R₇ is a branched or straight-chained C₁₀-C₈₀₀ alkyl or alkenyl group;
wherein the N atom bound to the R₂ and R₃ groups above is optionally be substituted in one or more places with the group:
—R₈—R₉
wherein R₈ is a C₁-C₁₀ branched or straight chained alkylene group; and R₉ is NH₂ or 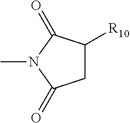
wherein R₁₀ is a branched or straight-chained C₁₀-C₈₀₀ alkyl or alkenyl group; and
wherein the R₂—NH—R₃ group is optionally be interrupted in one or more places by a heterocyclic or homocyclic cycloalkyl group; and
(b) a boronating agent associated or complexed with the additive defined in section (a).

19. The composition of claim 18, wherein the boronating agent is selected from boric acid, an ortho-borate, or a meta-borate.

20. The composition of claim 19, wherein the boronating agent is selected from boric acid, trimethyl metaborate (trimethoxyboroxine), triethyl metaborate, tributyl metaborate, trimethyl borate, triethylborate, triisopropyl borate (triisopropoxyborane), tributyl borate (tributoxyborane) and tri-t-butyl borate.

21. The composition of claim 20, wherein the additive is selected from: 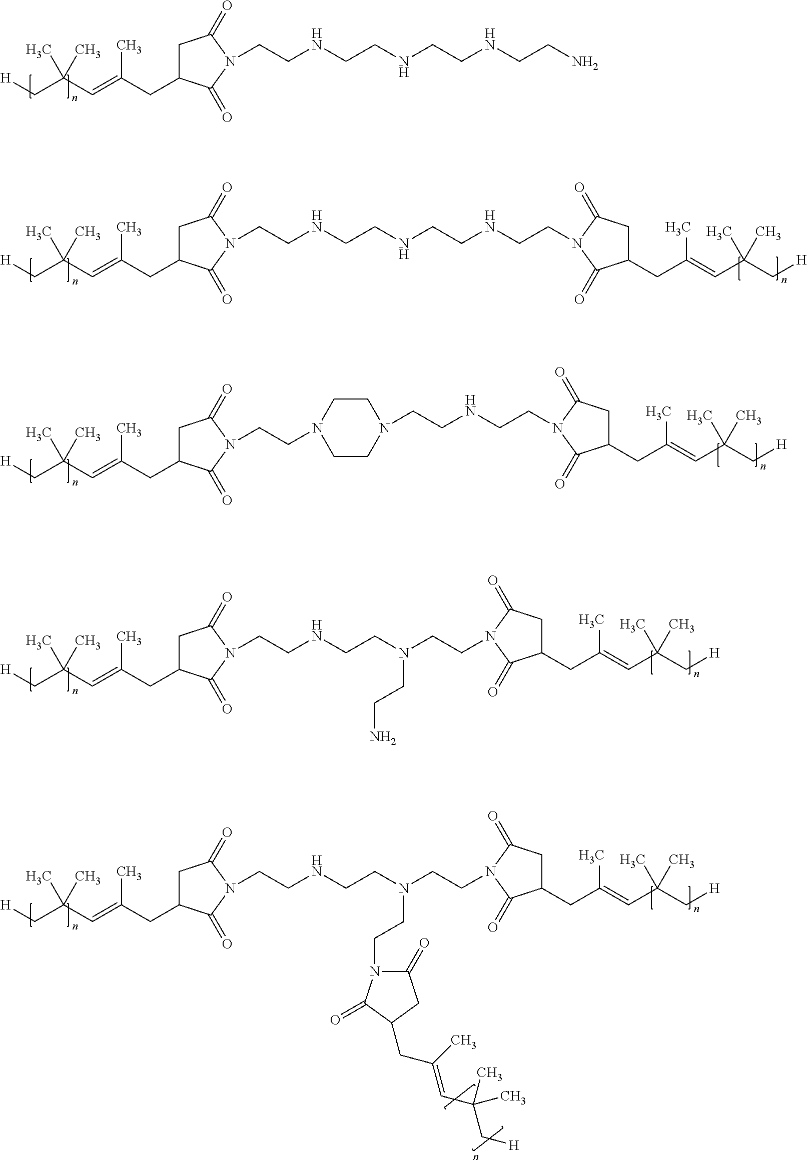
wherein n independently from 1 to 500 inclusive.

22.-27. (canceled)

28. The composition of claim 18, wherein the additive is selected from: 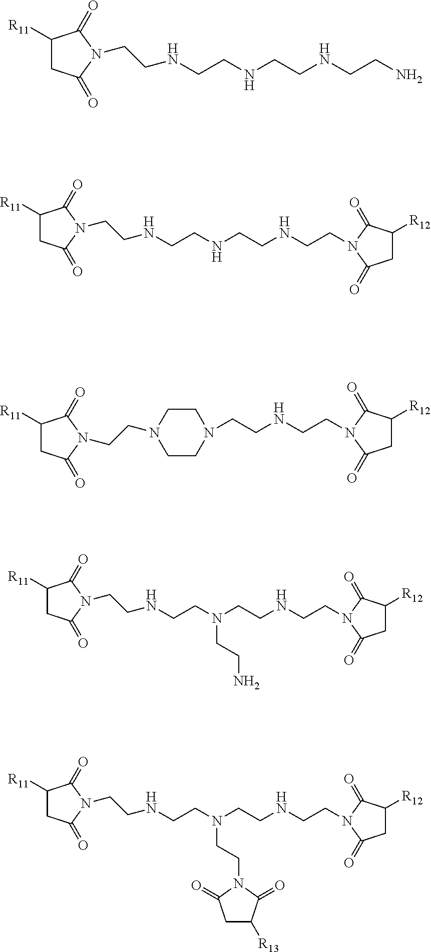
wherein R₁₁, R₁₂, and R₁₃ independently comprise a polymer selected from polypropylene and poly(ethylene-co-propylene).

29. A compound useful for antifouling of a crude oil refining process, prepared by the method comprising:
(a) reacting polypropylene having a number-averaged molecular weight of about 300-30,000 g/mol and having an allylic vinyl terminal group with maleic anhydride;
(b) reacting the product formed in (a) with a polyamine represented by 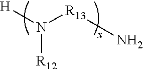
wherein R₁₂ is hydrogen or a C₁₋₁₀ branched or straight chained alkyl optionally substituted by one or more amine groups, R₁₃ is a C₁-C₁₀ branched or straight chained alkylene group, and x is an integer between 1 and 10 inclusive.

30. A compound useful for reducing fouling in a crude oil refining process, prepared by the method comprising:
(a) reacting poly(ethylene-co-propylene) having a number-averaged molecular weight of about 300-30000 g/mol and having an allylic vinyl terminal group with maleic anhydride;
(b) reacting the product formed in (a) with a polyamine represented by 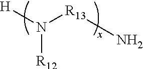
wherein R₁₂ is hydrogen or a C₁₋₁₀ branched or straight chained alkyl optionally substituted by one or more amine groups, R₁₃ is a C₁-C₁₀ branched or straight chained alkylene group, and x is an integer between 1 and 10 inclusive.

31. The compound of claim 30, wherein the poly(ethylene-co-propylene) contains about 1 to about 90 mole % of ethylene units and about 99 to about 10 mole % propylene units.
